(19) United States
(12) Patent Application Publication
(10) Pub. No.: US 2025/0299329 A1
(43) Pub. Date: Sep. 25, 2025

(54) COMPUTER-IMPLEMENTED METHOD AND APPARATUS FOR COMPARING IMAGES

(71) Applicant: Siemens Healthineers AG, Forchheim (DE)

(72) Inventors: Martin KRAUS, Fuerth (DE); Mehmet Akif GULSUN, Princeton, NJ (US); Max SCHOEBINGER, Hirschaid (DE); Michael WELS, Bamberg (DE)

(73) Assignee: Siemens Healthineers AG, Forchheim (DE)

(21) Appl. No.: 19/082,843

(22) Filed: Mar. 18, 2025

(30) Foreign Application Priority Data
Mar. 19, 2024 (DE) 10 2024 202 561.5

(51) Int. Cl. G06T 7/00; G06T 7/33; G06V 10/25; G06V 10/426; G06V 10/74; G16H 30/40

(52) U.S. Cl. CPC G06T 7/0012; G06T 7/33; G06V 10/25; G06V 10/426; G06V 10/761; G16H 30/40; G06T 2207/30096; G06T 2207/30101

(57) ABSTRACT

A method for comparing images, comprises: receiving images with the same subject matter that have been recorded at different times; establishing structures in the images and generating directed acyclical graphs based on the structures in the images, wherein each graph has specified points; registering a graph of at least one second image to the graph of a first image; establishing a correspondence between the points of the registered graphs, based on the spatial proximity of the points in conjunction with a link structure of the graphs; registering at least regions of the images that are specified by corresponding points, according to the registered graphs; and outputting at least the registered regions of the images.

### CROSS-REFERENCE TO RELATED APPLICATION(S)

The present application claims priority under 35 U.S.C. § 119 to German Patent Application No. 10 2024 202 561.5, filed Mar. 19, 2024, the entire contents of which is incorporated herein by reference.

### FIELD

One or more embodiments of the present invention relate to a computer-implemented method and an apparatus for comparing images, a control facility (control device) for controlling a medical technology system, in particular a diagnostic system or an imaging system and a medical technology system.

### BACKGROUND

In computed tomography (CT) angiography (CTA), CT recordings of vessels, for example, of the coronary vessels of a patient, are prepared and subsequently assessed, often over a period of time. Therein, it is often not only 3D regions that are to be compared using individual 3D points, but rather compact regions are also of interest. Such regions are, for example, diseased regions or regions with stents in the course of the vessels.

For particular applications such as, for example, multiphase CTA, typically, correspondences between points on segmented vascular trees from one phase to the other are sought. This can be achieved with the aid of a registration algorithm which operates either on the fundamental images or on segmented anatomical structures such as midline trees of vessels. The registration can be either rigid or elastic.

Known automated landmark-based registration methods are very fault-prone and sometimes false routes of vessels are shown. This is very disadvantageous during a subsequent diagnosis by a person.

### SUMMARY

It is an object of the present invention to provide a method and an apparatus for comparing images, a control facility (also referred to as a control device) for controlling a medical technology system and a medical technology system with which the aforementioned disadvantages can be avoided.

At least this object is achieved at least by way of a computer-implemented method, an apparatus, a control facility (control device) and/or a medical technology system as claimed.

A computer-implemented method, according to an embodiment of the present invention, serves for (in particular, automatic) comparison of images. It comprises the following steps:
receiving images with the same subject matter that have been recorded at different times,
establishing structures in the images and generating directed acyclical graphs on the basis of the structures in the images, wherein each graph has specified points,
registering a graph of at least one second image to the graph of a first image,
establishing a correspondence between the points of the registered graph, on the basis of the spatial proximity of the points in conjunction with a link structure of the graphs,
registering at least regions of the images that are specified by way of corresponding points according to the registered graphs and output of at least the registered regions of the images,
optionally: comparing regions of the images that are specified by way of corresponding points.

With this procedure, a fully automatic matching can be achieved which links 3D time points, in particular of 3D-CCTA scans via suitable interim geometrical representations. The representations arising therefrom can encode, for example, the coronary topology, in particular successively branched vessels, in a graph-theory tree. This enables the allocation of compact regions, which, in turn, can be used to provide intuitive side-by-side views of these regions from a first examination and from subsequent examinations, for example, with regard to lesion-focused curved planar views.

The method is typically carried out with medical images of vessels, in particular with images of coronary vessels. Even though the method is readily usable in two dimensions, it is preferred that the images are three-dimensional images, for example, CT images. The method is particularly advantageous for photon counting CT (PCCT). The method preferably serves for an automated comparison of images, in particular of medical follow-up examinations, since changes in vessels over time can be acquired very well.

Firstly, the images are captured. For a good understanding of the subsequent steps, it should be understood that these are 3D CT images. The images can have been recorded in advance and/or made available in an image database.

The images should show the same subject matter, that is, the same vessel structure. “The same subject matter” means that it can be the same subject matter (that is, the corresponding vessel structure of the same patient), but also similar subject matter, that is, the corresponding vessel structure in different patients. The method is both advantageous for a representation of a change in a vessel structure of a patient and also for a comparison of corresponding vessel structures of a plurality of patients. For a good understanding of the following, it can be imagined that the coronary arteries of a patient are observed over a period of time (across a plurality of examinations). A possible time course is the different heart phases during a cardiac cycle, including in the setting of a single examination.

Once the images have been captured, structures (that is, in principle, the vessels, in particular blood vessels) are established in these images. On the basis of the established structures, directed acyclical graphs (that is, essentially, vascular trees) are then generated in the images. A directed graph is known from the prior art and comprises a quantity of nodes and a quantity of edges which each connect node pairs to one another. The edges are directed edges which can only be traversed in one direction. A directed acyclical graph is itself a directed graph which contains no directed cycles. This illustrates an important point of one or more embodiments of the present invention, since the limitation of the possible results to such graphs (in particular to tree structures) eliminates a series of potential errors. In the following, a tree structure can be imagined as a graph for vessels.

Each graph has a plurality of specified points. These serve to enable the graphs of different images to be compared with one another. Theoretically, the nodes could simply be regarded as points, that is, always with a point placed at a branching of a structure. However, this is problematic in practice since in different images, not every branching is always reliably recognizable as such. It is therefore preferred to place points on the edges (possibly in addition to recognized nodes, which preferably here also represent a point). The spacing of the points is preferably less than 1 cm, particularly preferably less than 3 mm. Since it is usually voxels that are used in digital images, the minimum spacing between two points is preferably less than 100 voxels, in particular less than 50 voxels, or even less than 10 voxels (the same applies for a spacing in pixels for pixel-based images).

It is particularly preferred that a granularity of points that has been applied in one image for a graph is also transferred to the other graphs of the other images. Thus, the points preferably have substantially the same granularity in all graphs and/or substantially the same spacing. The expression “substantially” here means “with a deviation of not more than 30%, in particular not more than 10%”.

In a further step, the graphs of two or more images are registered to one another. For this purpose, one of the images is preferably selected and the graphs of the other images are registered to the graph of this image. This can come about in that the images (together with the graphs or before the generation of the graphs) are registered to one another, but it is preferred that only the graphs are registered to one another, which compared with a complete image registration requires a much smaller computation effort. The fundamental principle of an image registration is known from the prior art. Preferably, the registration is based upon an elastic iterative algorithm for the respective nearest point with one or more regularizing terms which penalize deformation, stretching/shrinkage of distances between successive tree points. Preferably, additionally existing vessel designations or other attributes are used as orientation for the registration.

It should be noted that after the registration, the aforementioned points do not necessarily have to lie upon one another. In practice, it will only be the case in the rarest cases that (despite an optimum registration), two points arbitrarily lie on the same position. This is attributable to changes and the movement of the vessels as well as to errors in the image recording.

Now, the points are nevertheless placed in connection with one another. This is also possible in all cases where the points do not lie exactly on the same site. If, for example, points were only set on the branchings of a graph, then (in ideal images) they would also match the corresponding branchings of the vessels and would correlate to one another even if the vessels were displaced. In the particularly preferred case in which (in particular, additionally) points on the edges are observed, junctions cannot also be recognized. In the case, for example, of points positioned close to one another, a correspondence could then be suspected. Precisely how this can be achieved is described in greater detail below. Essentially, “correspondence” means that corresponding points lie on the same edge and/or in the same node. This correspondence is thus established based upon the spatial proximity of the points in conjunction with the link structure of the graphs. The link structure corresponds to the topology of the links of a graph, that is, how the graph is distributed in its limbs. Thus, the particular structure of the graphs (e.g. the shape of a tree) directly affects the correspondence.

If now a region of interest (ROI) is sought in the one image, lie in the points of the graphs there. Then, the points corresponding to these points can be sought in the other graphs.

The original images, but at least their regions that are specified by corresponding points, are then (for a comparison) registered to one another. So that the structures, for example vessels, match and/or are comparable, the registration of the graphs is used for this purpose. The registration of the regions therefore takes place according to the registered graphs, that is, the relevant region of a structure (e.g. a vessel) is exactly as deformed as its graph at this site. Since for later comparisons, only the structures are relevant, it is fundamentally unimportant how the regions of the images are registered outside the structures. For the structures themselves, the registration of the graphs applies. The registered regions (and/or the entire images) are then output, for example, by storing or by display for a subsequent comparison.

It should be noted that with this registration, the fundamentals for a comparison are already established, since the regions that are mutually matched thereby can be compared with one another at a glance. However, even if a visual comparison is possible, it is still preferable to undertake an automated comparison which assists the work of an evaluator.

During a comparison, the region of one of the other images that is compared with the region of the first image is then determined by way of the relevant corresponding points. If, therefore, the ROI in an image is the region of a stent or a narrowing, it would be checked which points of the graph lie there, the corresponding points of the other graphs established, the respective images checked where these points lie therein and these regions in the other images assumed to be those where the ROI is situated.

The fundamental concept of one or more embodiments of the present invention is that in each image, that is, at different recording time points, vascular trees are segmented along the midline of vessels from 3D images, then the vascular trees are registered with one another, and then correspondences between points on these vascular trees are established. For this purpose, following the registration, a step for creating the correspondence is preferably then carried out. For this, in particular, the point proximity is used in order to create correspondences and simultaneously to ensure that the resulting double tree structure with links does not have any cycles.

An apparatus, according to an embodiment of the present invention, serves for (in particular, automatic) comparison of images. It comprises the following components:
a data interface configured for receiving images of the same subject matter that have been recorded at different times,
an establishing unit configured to establish structures in the images and to generate directed acyclical graphs on the basis of the structures in the images, wherein each graph has specified points,
a registration unit configured to register a graph of at least one second image to the graph of a first image,
a correspondence unit configured for establishing a correspondence between the points of the registered graph, on the basis of the spatial proximity of the points in conjunction with a link structure of the graphs,
a comparison unit configured for registering at least regions of the images that are specified by way of corresponding points, according to the registered graphs and output of at least the registered regions of the images, and preferably also for comparison of regions of the images that are specified by the corresponding points.

The function of the components of the apparatus has already been described above. The apparatus is preferably configured for carrying out a method, according to an embodiment of the present invention. As far as the “comparison unit” is concerned, this could also be referred to as a “second registration unit” or “computing unit”. The name is intended here to recall the fact that only the specific registration of the image regions enables an improved comparison of the structures.

A control facility (also referred to as a control device), according to an embodiment of the present invention, serves to control a medical technology system, in particular, a diagnostic system and/or imaging system. It comprises an apparatus, according to an embodiment of the present invention, and/or is configured to carry out a method, according to an embodiment of the present invention.

A medical technology system, according to an embodiment of the present invention, is preferably a diagnostic system or an imaging system and comprises a control facility, according to an embodiment of the present invention.

One or more embodiments of the present invention can be realized, in particular, in the form of a computer unit with suitable software. For this purpose, the computer unit can have, for example, one or more cooperating microprocessors or the like. In particular, it can be realized in the form of suitable software program parts in the computer unit. A realization largely through software has the advantage that conventionally used computer units can also easily be upgraded with a software and/or firmware update in order to operate in the manner according to embodiments of the present invention. The object is therefore also achieved, in particular, with a corresponding computer program product having a computer program which can be loaded directly into a memory facility (also referred to as a memory or memory device) of a control unit, having program portions in order to carry out all the steps of the method, according to an embodiment of the present invention, when the program is executed in the computer unit. Such a computer program product can comprise, where relevant, apart from the computer program, additional constituents, such as, for example, documentation and/or additional components, and also hardware components, for example, hardware keys (dongles, etc.) in order to use the software.

For transport to the computer unit and/or for storage at or in the computing unit, a computer-readable medium, for example a memory stick, a hard disk or another transportable or firmly installed data carrier can be used on which the program portions of the computer program which can be read in and executed by a computer unit are stored.

Further particularly advantageous embodiments and developments of the present invention are disclosed in the dependent claims and the following description, wherein the claims of one claim category can also be further developed similarly to the claims and description passages relating to another claim category and, in particular also, individual features of different exemplary embodiments and/or variants can be combined to new exemplary embodiments and/or variants.

According to a preferred embodiment of the method, the inclusion of the link structure of the graphs in the establishment of a correspondence in at least two steps takes place, at least with the steps:
a) establishing a correspondence between limbs of the registered graphs, preferably wherein groups of points are established on the respective limbs and correspondences are generated between these groups, and
b) establishing a correspondence between the points of groups of mutually matching limbs of the registered graphs, preferably wherein for this purpose, exclusively the points of the matching groups are considered.

A preferred possibility for creating correspondences lies in initially linking branches to one another. A limb is the route from a distal point of a graph (e.g. a tree) in the direction of its roots. For this, a spacing measure between branches can preferably be calculated. Based on the assumption that xi and yj are the registered points of two branches, the spacing can be expressed as

$${\sum }_{i , j} {min}_{i} ( \vert {x}_{i} - {y}_{j} \vert ) + {\sum }_{i , j} {min}_{j} ( \vert {x}_{i} - {y}_{j} \vert ) .$$

The two sums of the formula provide the symmetry of the expression. In a spacing measure of this type, further information such as the segment labels of the branches and their difference can be included.

Preferably, the inclusion of the link structure of the graphs in the establishment of a correspondence comprises a check of whether, by way of the correspondence, a cycle arises in a resulting graph, wherein a correspondence with a cycle is declined. Since the graphs are acyclical by definition, a correspondence that yields a cycle cannot be correct. In this way, prior knowledge of vessel structures can directly enable a reduction of errors.

Preferably, the inclusion of the link structure of the graphs in the establishment of a correspondence comprises a check of hierarchies of the points, wherein a correspondence with a hierarchy that does not comply with the rule of a directed graph is declined. In this way also, prior knowledge of vessel structures can directly enable a reduction of errors. If, for example, an undirected graph results, a correspondence can be rejected as faulty and a new correspondence can be sought.

Preferably, the inclusion of the link structure of the graphs in the establishment of a correspondence comprises a check of whether, by way of the correspondence, a topology arises that matches neither of the two graphs and, in this case, a correspondence is declined.

Preferably, the inclusion of the link structure of the graphs in the establishment of a correspondence comprises a comparison of points on corresponding limbs of graphs on a route from proximal to distal and/or on a route from distal to proximal. Given inconsistencies, a correspondence is preferably declined.

Given all these alternatives which can be applied alone or in combination with one another, prior knowledge of the graphs and/or the strict definition of the graphs as directed and acyclical reduces possible errors. In this way, correspondences that are recognized as faulty can be declined and new, error-free correspondences can be sought.

Preferably, to establish a correspondence between points (a point in each graph is meant in each case, but also groups of points, e.g. limbs), a distance matrix is established and a branching comparison is derived therefrom. It is preferable therein that in the corresponding graphs, a spacing measure between pairs of points is calculated and, starting from a plurality of distance measures of point pairs, this distance matrix is calculated. Alternatively or additionally, it is also preferred that the branching comparison is derived via the Hungarian method (also called the “Kuhn-Munkres algorithm”).

Starting from a distance measure (between points or whole graphs), said distance matrix can be calculated in that all the pairwise distances are evaluated. With the aid of the Hungarian algorithm, a matching between branches of the graphs can be calculated efficiently. Following a comparison of the branchings, correspondences between points can then be created.

In particular, the limbs of the first graph can be searched through in a particular sequence, for example, by decreasing significance or length of the branch. Where the first branch is specified, the second branch is defined by the branch match. With the aid of the Hungarian algorithm, points of the two branches that do not yet match can be further compared. Herein, it should be noted however that, for example, on a transition from distal to proximal in the first graph (tree), the sequence of the matchings in the second graph also takes place from distal to proximal.

Furthermore, the addition of a correspondence between two points can lead to conflicts with previously added points. In such cases, the correspondence should not be added.

Here also, further conflict solving strategies could advantageously be applied, such as backtracking and randomizing the sequence in which branches are processed. As soon as the points of two branches match, the process continues with the next branch.

This results in a series of correspondences that can be further refined. For example, only a subset of the points of both trees have been compared, which could lead to a sparse match (only a few points). This sparse matching can be made denser by adding correspondences between further points. Traveling along a limb from distal to proximal, the points on both limbs can again be compared between a first and a second correspondence (that is, between corresponding point pairs), so that denser correspondences come about. This can also be carried out, for example, with the Hungarian algorithm. The sequence of the points should be maintained in both branches.

According to a preferred embodiment of the method, therefore, following a first establishment of a correspondence between points, a further correspondence is carried out between points for which after the first establishment, no correspondence has yet been able to be created, and/or which have subsequently been inserted into the graphs.

On the basis of these correspondences, ROIs from a first image and therefore also from a time point, are automatically assigned to one or more regions of other images (i.e. other time points). The regions in the other images correspond to the ROI in the first image. If the ROI is, for example, a coronary artery lesion, the attributes of the lesion can be set in relation to other time points (images) and collected in an overview. For example, each lesion has a maximum stenosis level. For the assessment of the disease progression, the tendency of the maximum stenosis level over time is relevant. Therefore, with existing conformities and midline vascular tree points (that is, points of the graphs), the following steps are preferred:
1. For a plurality of points (in particular, for each point) in the ROI of a first image: establishing corresponding points in at least one other image,
2. Establishing values for a number of parameters of the ROI from the points in the first image, e.g. establishing the maximum stenosis level,
3. Extracting values of the same parameters and/or the same parameter from the corresponding points of the at least one other image,
4. Bringing the values into relation, in particular during the temporal course of the recording of the images in order to demonstrate a progressing course.

This approach can be used alongside the example of a stenosis, in particular also for monitoring the progression of the plaque build-up, the plaque composition or the susceptibility to plaque and for an assessment after a PCI (percutaneous coronary intervention), for example, of the FFR (fractional flow reserve).

Furthermore, corresponding portions of points in 3D volumes of the ROI of the images (corresponding to different time points) can be visualized, for example, with curved planar reformation (CPR). For each time point, additional attributes such as start and stop markers for a lesion can thus be displayed. These can be mapped via suitable points from the lesion at a first time point (image from an examination) and subsequent time points (images of further examinations). Alternatively and additionally, the other lesions of the quantity of relevant points can also have zero or more lesion regions. These markings can also be visualized at the respective time point.

This automated processing can also be used in order to generate an overview report regarding changes, e.g. lesions, and their progression status. There, the changes can be sorted, for example, according to the size of a progression between time points. An advance can be displayed visually, for example, with an equals sign, an upward arrow and a downward arrow in order to represent constancy, an increase or a decrease.

Such automatically obtained progression information items can also be used for the creation of cardiological reports.

Preferably, the graphs extend as lines in the structures. Therein, the structures are preferably vessels in a body, in particular blood vessels, and the directed acyclical graphs are vascular trees in the vessels. Preferably, they extend along the midline of the vessels in the images.

Preferably, the registration of graphs to one another is based upon an elastic iterative algorithm for the respective nearest point with one or more regularizing terms which load the deformation, stretching/shrinkage of distances between successive tree points with a penalty value. Preferably, labels of a segmentation and/or further information relating to the structures are additionally used as orientation for the registration.

A preferred embodiment of the method comprises the following steps for a comparison of regions of the images (that is, from different time points):
selection of a region of interest in a first image, preferably a region with a fault in the structure, in particular, a lesion, a stent, a stenosis, plaque or a region of a percutaneous coronary intervention,
specifying the points of the graph in the first image which lie within this region,
establishing the corresponding points in a number of further images and, in each case, specifying a region in the number of further images that is predetermined by these corresponding points,
comparing the specified regions with the region of interest (ROI) in the first image, preferably wherein a parameter value of the region of interest is established and a number of corresponding parameter values is derived from the number of specified regions and these parameter values are compared with one another,
optionally: establishing the recording time points of the images and establishing a course while taking account of the recording time points.

It is therein preferred that for a visualization of establishing results, points are visualized via a curved planar reformation.

According to a preferred embodiment of the method, the correspondence between the points of the registered graphs is additionally used to place image points of the images that lie outside the structures in relation to one another, so that corresponding regions result outside the structures. It is therein preferred that these corresponding regions are compared with one another.

A preferred apparatus is distinguished in that the correspondence unit is configured to establish firstly a correspondence between limbs of registered graphs and then to establish a correspondence between the points of groups of mutually corresponding limbs of the registered graphs.

During the establishing of a correspondence between limbs of registered graphs, it is preferable that in the graphs, a spacing measure between pairs of groups of corresponding limbs is calculated and, starting from a plurality of distance measures of pairs, a distance matrix is calculated and therefrom a branching comparison is derived.

During the establishing of a correspondence between the points of groups of mutually corresponding limbs of the registered graphs, it is preferable that in the graphs, a spacing measure between pairs of points of corresponding groups is calculated and, starting from a plurality of distance measures of pairs, a distance matrix is calculated and therefrom a branching comparison is derived.

Preferable is the use of AI (artificial intelligence)-based methods for the method according to one or more embodiments of the present invention. An artificial intelligence system is based on the principle of machine-based learning and is typically carried out with a learning-capable algorithm that has been suitably trained. For machine-based learning, the expression “machine learning” is often used, which here also includes the principle of “deep learning”.

Preferably, components of embodiments of the present invention are present as a “cloud service”. A cloud service of this type serves for processing data, in particular, via an artificial intelligence, but can also be a service on the basis of conventional algorithms or a service in which an evaluation by humans takes place in the background. In general, a cloud service (also designated “cloud” in the following) is an IT infrastructure in which, for example, storage space or computing power and/or an application software is made available via a network. The communication between the user and the cloud takes place via data interfaces and/or data transfer protocols. In the present case, it is particularly preferred that the cloud service makes both computing power and also application software available.

In the context of a preferred method, a provision of data that is obtained in the context of the present invention, takes place via the network to the cloud service. This comprises a computer system which typically does not include the local computer of the user. The method can therein be realized via a command combination in a network. The data calculated in the cloud is preferably transferred again later via the network to the local computer of the user.

### BRIEF DESCRIPTION OF THE DRAWINGS

The present invention will now be described again in greater detail using exemplary embodiments, making reference to the accompanying drawings. In the various drawings, the same components are provided with identical reference signs. The drawings are, in general, not to scale. In the drawings:

FIG. 1 shows a rough schematic representation of a CT system with an exemplary embodiment of a control facility or device according to the present invention for carrying out the method,

FIG. 2 shows an image of a vessel with a vascular tree,

FIG. 3 shows a block diagram of the sequence of the method,

FIG. 4 shows an overview of the effect of the method.

### DETAILED DESCRIPTION

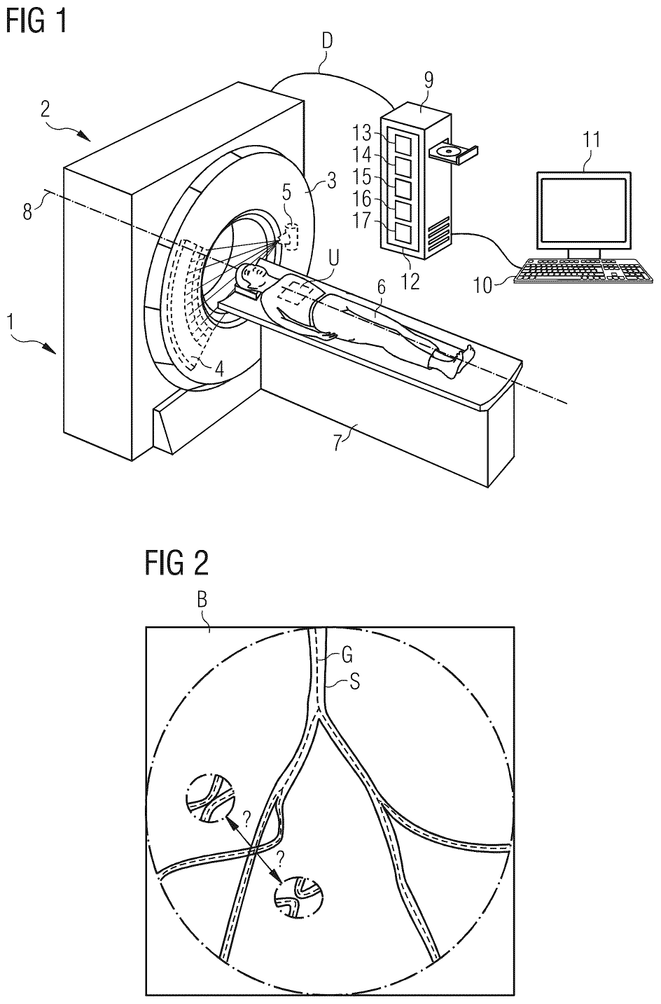

FIG. 1 shows an embodiment of a computed tomography (CT) system 1 with a radiation detector 4 and a radiation source 5. The radiation source 5 is configured to irradiate the radiation detector 4 with radiation. The CT system 1 shown comprises a gantry 2 with a rotor 3. The rotor 3 comprises, as the radiation source 5, an X-ray source 5, and the radiation detector 4 which is designed to detect X-ray radiation.

The rotor 3 is rotatable about the rotation axis 8. The patient 6 is positioned on the patient support 7 and is able to be moved along the rotation axis 8 through the gantry 2. The head of the patient 6 is supported on a positioning aid L. In order to control the imaging system 1 and/or to generate an X-ray image dataset on the basis of signals detected by the radiation detector 4, the computing unit 9 is provided, which is connected via a data line D to the gantry 2.

Typically a (raw) X-ray image dataset of the examination object 6 is recorded from a large number of angular directions via the radiation detector 4 at one radiation energy, and therefore two or more raw datasets. Subsequently, on the basis of the (raw) X-ray image dataset, via a mathematical method, for example comprising a filtered back projection or an iterative reconstruction method, a (final) X-ray image dataset can be reconstructed.

The computing unit 9 serves here as a control facility 9 (also referred to as control device 9) for controlling the CT system 1. An input facility 10 (also referred to as an input device 10) and an output facility 11 (also referred to as an output device 11) are connected to this computing unit 9. The input facility 10 and the output facility 11 can, for example, enable an interaction by way of a user or the representation of a generated image dataset B.

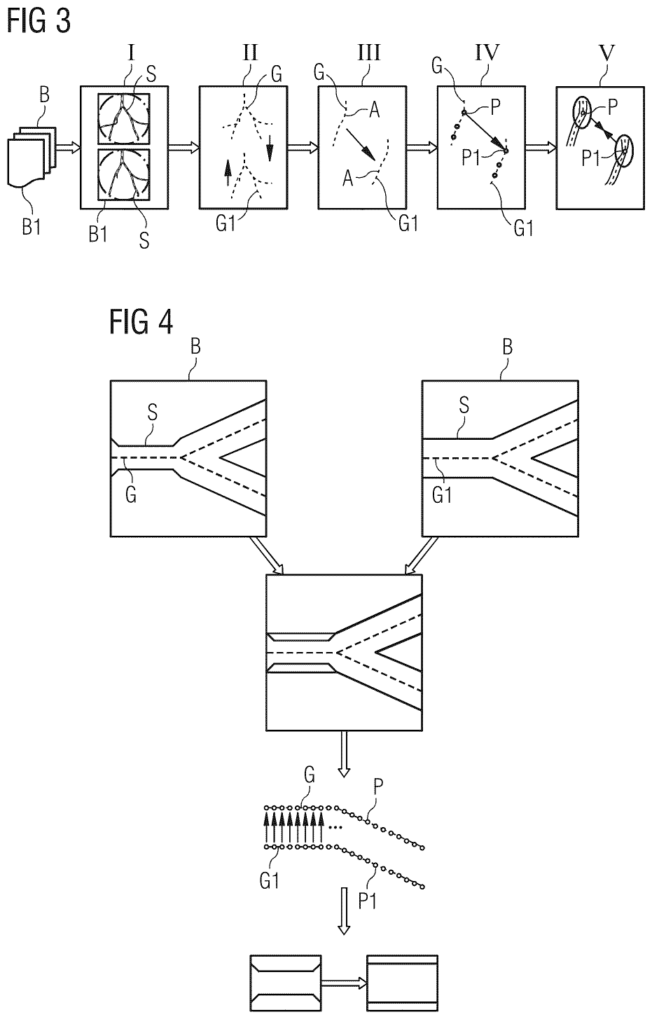

The control facility 9 comprises an apparatus 12, according to an embodiment of the present invention, for comparing images B, B1 in accordance with a method, according to an embodiment of the present invention (see FIG. 3). The apparatus 12 comprises a data interface 13, an establishing unit 14, a registration unit 15, a correspondence unit 16 and a comparison unit 17.

The data interface 13 serves for receiving images B, B1 of the same subject matter that have been recorded at different times by the CT system. It should be noted that the apparatus 12 is also advantageous in a diagnostic system. However, the example of a CT system 1 has been given here in order also to take account of the recording of the images B. For example, the control unit 9 registers the identity of the patient 6 currently being examined and automatically searches for images B1 from previously performed examinations of this patient 6.

The establishing unit 14 serves for establishing structures in the images B, B1 and for generating directed acyclical graphs G, G1 on the basis of the vessels represented in the images B, B1, wherein each graph G, G1 has specified points P, P1.

The registration unit 15 serves for registering a graph G, G1 of at least one second image B1 to the graph of a first image B.

The correspondence unit 16 serves for establishing a correspondence between the points P, P1 of the registered graph G, G1, on the basis of the spatial proximity of the points P, P1 in conjunction with the link structure of the graphs G, G1.

The comparison unit 17 serves for registering at least regions of the images B, B1 that are specified by way of corresponding points P, P1 according to the registered graphs G, G1 and output of at least the registered regions of the images B, B1. It serves here also for comparing regions of the images B, B1 that are specified by way of corresponding points P, P1.

FIG. 2 shows an image B of a vessel S with a vascular tree G. This vascular tree G is a directed acyclical graph G. It can be seen that the vascular tree G extends primarily in the center of the vessel S. Purely visually, it is simple to follow the course of the graph. However, if it were assumed that each of the dashes were to represent a point, then in the region of the crossing on the left side, an error could arise that the crossing is regarded as a convergence.

In circles above and below the crossing point, potential misinterpretations are shown, which could possibly be recognized as corresponding points (question marks indicate the possibilities and the uncertainty).

Whereas the misinterpretation over the crossing point would be an error that can be prevented by labeling the limbs of the graph, the lower misinterpretation can be excluded purely in that the graph is not permitted to be cyclical.

FIG. 3 shows a block diagram of the sequence of the method and FIG. 1 shows a method for comparing images.

In step I, 3D images B, B1 with the same subject matter are received, having been recorded at different times.

In step II, an establishing of blood vessels S as structures S in the images B, B1 and the generating of directed acyclical graphs G, G1 takes place on the basis of the structures in the images B, B1. Each graph G, G1 therein has specified points P, P1.

In step III, a registration of the graph G1 of at least one second image B1 to the graph G of the first image B takes place. Shown here, by way of substitute are corresponding limbs A of the graphs G, G1.

In step V, an establishing of a correspondence takes place between the points P, P1 of the registered graphs G, G1, on the basis of the spatial proximity of the points P, P1 in conjunction with the logical structure of the graphs G, G1.

In step VI, a registration takes place of at least regions of the images B, B1 that are specified by way of corresponding points P, P1 according to the registered graphs G, G1 and output of at least the registered regions of the images B, B1, and a comparison of regions of the images B, B1 which are specified by way of corresponding points P, P1.

FIG. 4 shows an overview of the effect of the method. At the top, two images B, B1 are shown, each showing the same vessel S. On the left, it can be seen that it has a constriction but not on the right. Now, the two images B, B1 are registered to one another and thus also the two graphs G, G1. This takes place here in order to represent the region of the constriction more clearly. In practice, it is simpler to register the two graphs G, G1 directly to one another.

Thereunder, the search for corresponding points P, P1 is represented. They lie very close to one another and the closest-lying points are assigned to one another as mutually corresponding. Now, the corresponding regions of the images B, B1 can be selected and compared (representation at bottom).

Finally, it should again be noted that the present invention described above in detail merely involves exemplary embodiments which can be modified by a person skilled in the art in a wide variety of ways without departing from the scope of the present invention. Furthermore, the use of the indefinite article “a” or “an” does not preclude the possibility that the relevant features can also be present plurally. Similarly, expressions such as “unit” do not preclude the relevant components consisting of a plurality of cooperating sub-components which can also be spatially distributed, if relevant. The expression “a number” is to be understood as meaning “at least one”.

Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility, data processing device, or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for comparing images, the computer-implemented method comprising:
receiving images with the same subject matter and that have been recorded at different times;
establishing structures in the images and generating directed acyclical graphs based on the structures in the images, wherein each directed acyclical graph has specified points;
registering a directed acyclical graph of at least one second image to a directed acyclical graph of a first image;
establishing a correspondence between the specified points of the registered directed acyclical graphs, based on a spatial proximity of the specified points in conjunction with a link structure of the directed acyclical graphs;
registering at least regions of the images that are specified by way of corresponding points according to the registered directed acyclical graphs; and
outputting at least the registered regions of the images.

2. The computer-implemented method according to claim 1, wherein inclusion of the link structure of the directed acyclical graphs in the establishing of the correspondence comprises:
establishing a correspondence between limbs of the registered directed acyclical graphs; and
establishing a correspondence between the specified points of groups of mutually matching limbs of the registered directed acyclical graphs.

3. The computer-implemented method according to claim 1, wherein inclusion of the link structure of the directed acyclical graphs in the establishing of the correspondence comprises at least one of:
checking, by way of the correspondence, whether a cycle arises in a resulting directed acyclical graph, and declining the correspondence with the cycle;
checking hierarchies of the specified points, and declining a correspondence with a hierarchy that does not comply with a rule of a directed acyclical graph;
checking, by way of the correspondence, whether a topology arises that matches neither of the registered directed acyclical graphs, and in this case, declining the correspondence; or
comparing points with one another on corresponding limbs of registered directed acyclical graphs on at least one of a route from proximal to distal or a route from distal to proximal and, declining a correspondence in response to inconsistencies in the comparing.

4. The computer-implemented method as claimed in claim 1, the establishing a correspondence between specified points comprises:
establishing a distance matrix; and
deriving a branching comparison from the distance matrix.

5. The computer-implemented method as claimed in claim 1, wherein following a first establishment of a correspondence between specified points, the method comprises:
establishing a further correspondence between points for which, after the first establishment, at least one of no correspondence has yet been able to be created or have subsequently been inserted into the registered directed acyclical graphs.

6. The computer-implemented method as claimed in claim 1, wherein the directed acyclical graphs extend as lines in the structures, wherein the structures are vessels in a body and the directed acyclical graphs are vascular trees in the vessels and extend on the midline of the vessels in the images.

7. The computer-implemented method as claimed in claim 1, wherein registering of the directed acyclical graphs is based on an elastic iterative algorithm for a nearest point with one or more regularizing terms which load the deformation, stretching/shrinkage of distances between successive tree points with a penalty value.

8. The computer-implemented method as claimed in claim 1, further comprising:
selecting a region of interest in the first image, wherein the region of interest includes a lesion, a stent, a stenosis, or is a region of a percutaneous coronary intervention;
specifying points of the directed acyclical graph of the first image which lie within the region of interest;
establishing corresponding points in a number of further images, and specifying a region in each further image that is determined by these corresponding points; and
comparing the specified regions with the region of interest in the first image, wherein a first parameter value of the region of interest is established and a number of corresponding second parameter values are derived from the number of specified regions, and wherein the first parameter value and the corresponding second parameter values are compared.

9. The computer-implemented method as claimed in claim 1, wherein the correspondence between the specified points of the registered directed acyclical graphs is additionally used to place image points of the images that lie outside the structures in relation to one another, so that corresponding regions arise outside the structures.

10. An apparatus for comparing images, the apparatus comprising:
a data interface configured to receive images of the same subject matter and that have been recorded at different times;
an establishing unit configured to establish structures in the images and generate directed acyclical graphs based on the structures in the images, wherein each directed acyclical graph has specified points;
a registration unit configured to register a directed acyclical graph of at least one second image to a directed acyclical graph of a first image;
a correspondence unit configured to establish a correspondence between the specified points of the registered directed acyclical graphs, based on a spatial proximity of the specified points in conjunction with a link structure of the directed acyclical graphs; and
a comparison unit configured to register at least regions of the images that are specified by way of corresponding points, according to the registered directed acyclical graphs, and output at least the registered regions of the images.

11. The apparatus as claimed in claim 10, wherein the correspondence unit is configured to
establish a correspondence between limbs of the registered directed acyclical graphs, and
and establish a correspondence between the specified points of groups of mutually corresponding limbs of the registered directed acyclical graphs.

12. A control device for controlling a medical technology system, the control device comprising the apparatus as claimed in claim 10.

13. A medical technology system, comprising the control device as claimed in claim 12.

14. A non-transitory computer program product comprising commands that, when executed by a computer, cause said computer to carry out the computer-implemented method as claimed in claim 1.

15. A non-transitory computer-readable storage medium comprising commands that, when executed by a computer, cause said computer to carry out the computer-implemented method as claimed in claim 1.

16. The computer-implemented method of claim 1, further comprising:
comparing regions of the images that are specified by way of corresponding points.

17. The computer-implemented method as claimed in claim 4, wherein the branching comparison is derived via the Hungarian method.

18. The computer-implemented method as claimed in claim 7, wherein at least one of labels of a segmentation or further information regarding the structures are used as orientation for the registering.

19. The computer-implemented method as claimed in claim 8, further comprising:
establishing recording time points of the images; and
establishing a course while taking account of the recording time points.

20. The computer-implemented method of claim 19, wherein for a visualization of results of the establishing recording time points, the time points are visualized via a curved planar reformation.

21. The computer-implemented method as claimed in claim 9, wherein corresponding regions are compared with one another.

22. The computer-implemented method according to claim 2, wherein
groups of specified points are established on respective limbs and correspondences are created between the groups; and
in establishing the correspondence between the specified points of groups of mutually matching limbs of the registered directed acyclical graphs, the specified points of relevant groups are considered exclusively.

23. The computer-implemented method as claimed in claim 4, wherein the establishing a distance matrix comprises:
calculating a spacing measure between pairs of specified points; and
calculating the distance matrix starting from a plurality of distance measures of specified point pairs.

24. The apparatus of claim 10, wherein the comparison unit is configured to compare regions of the images which are specified by way of corresponding points.

25. The apparatus as claimed in claim 11, wherein the correspondence unit is configured to, in the directed acyclical graphs,
calculate a spacing measure between pairs of groups of corresponding limbs,
calculate a distance matrix starting from a plurality of distance measures of pairs, and
derive a branching comparison from the distance matrix.

26. The apparatus as claimed in claim 11, wherein the correspondence unit is configured to, in the directed acyclical graphs,
calculate a spacing measure between pairs of specified points of corresponding groups,
calculate a distance matrix starting from a plurality of distance measures of pairs, and
deriving a branching comparison based on the distance matrix.
